(19) United States
(12) Patent Application Publication
(10) Pub. No.: US 2005/0260606 A1
(43) Pub. Date: Nov. 24, 2005

(54) Use of whole blood in PCR reactions

(76) Inventors: Milko B. Kermekchiev, St. Louis, MO (US); Wayne M. Barnes, University City, MO (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(21) Appl. No.: 10/850,816

(22) Filed: May 20, 2004

(51) Int. Cl. C12Q 1/68; C12P 19/34

(52) U.S. Cl. 435/6; 435/91.2

(57) ABSTRACT

A method of obtaining DNA amplification of a nucleic acid target from a volume of whole blood comprising performing DNA amplification in a PCR assay mixture with a blood-resistant polymerase.

### BACKGROUND

The polymerase chain reaction (PCR) is a sensitive DNA amplification procedure that permits the selection and detection of specific nucleic acids from a complex mixture. In its most rudimentary form, PCR is employed using a sample that contains a target nucleic acid (DNA), a set of DNA primers that hybridize to the target, and a DNA polymerase that is capable of primer-based synthesis of complementary strands of the target. During the nucleic acid amplification process, the target:primer:polymerase mixture is subjected to successive rounds of heating at different temperatures to facilitate target DNA strand separation (performed at ˜90-99° C.), primer:target DNA strand annealing (performed at ˜40-70° C.), and DNA polymerase-mediated primer elongation (performed at ˜50-72° C.) to create new complementary target strands. Because the reaction may be subjected to ˜25-45 rounds of cycling to yield the desired DNA amplification product, PCR is usually conducted using thermal stable DNA polymerases that can withstand the very high temperatures associated with target strand separation without suffering inactivation due to heat-induced protein denaturation. Since its introduction in the mid-1980's, PCR has become the defacto standard for detecting minute quantities of nucleic acids in samples, and obtaining specific genes from complex DNA genomes and samples.

A major problem with diagnostic and forensic techniques based on PCR is the false-negative reactions or low sensitivity caused by inhibitory substances that interfere with PCR (1, 2, 3). Of particular clinical importance is the PCR analysis of blood samples, which represents the largest fraction of human health related tests for diagnosis of genetic diseases, virus and microbial infections, blood typing, and safe blood banking. Various studies indicate that the inhibitory effect of blood on PCR is primarily associated with direct inactivation of the thermostable DNA polymerase and/or capturing or degradation of the target DNA and primers. It has been reported that the protease activity in blood also contributes to the reduced efficiency of PCR (1-5, 7, 10, 12).

The blood-resistance characteristics of the thermostable DNA polymerases vary with the source of the enzyme (6). Widely used thermostable polymerases like Thermus aquaticus DNA polymerase (Taq) and AmpliTaq Gold are completely inhibited in the presence of 0.004-0.2% whole human blood (vol/vol; 3, 4, 6). Various agents have been tested for reducing the inhibitory effect of blood on Taq. It was found that an addition of betaine, bovine serum albumin, the single-stranded DNA binding protein of the T4 32 gene (gp 32), or a cocktail of protease inhibitors can partially relieve the blood inhibition and allow Taq to work in up to 2% blood (vol/vol), although this effect could be sample specific (3, 8, 9, 11).

Several major inhibitors of PCR in human blood have been characterized such as immunoglobulin G, hemoglobin, lactoferrin and excess of leukocyte DNA (4, 7, 10). The IgG, hemoglobin, and lactoferin have been purified from plasma, erythrocytes and leukocytes, respectively, using size-exclusion and anion-exchange chromatography (4, 7). The heme has been reported to inactivate the Taq polymerase by binding to its catalytic domain (10), while the mechanism of action of the other inhibitory components is more poorly understood. The inhibitory effect of IgG can be reduced when this plasma fraction is heated at 95° C. before adding it to PCR, or with the addition of excess non-target DNA to the PCR mixture. However, heating of IgG together with target DNA at 95° C. was found to block amplification. Inhibition by IgG may be due to an interaction with the single-stranded DNA fraction in the target DNA. The inhibitory effect could be removed also by treating the plasma with DNA-agarose beads prior to amplification (4).

Other complicating factors include EDTA and heparin, used as anti-coagulants, which can also inhibit DNA amplification. The addition of heparinase has been shown to counteract the heparin-mediated inhibition (13, 14). Therefore, various laboratory procedures of sample preparation have been developed to reduce the inhibitory effect of blood. The DNA purification methods suitable for PCR can include additional steps like dialysis, treatment with DNA-agarose beads or Chelex 100 resin, multiple DNA washes, or a combination of dilution with buffer which causes lysis of red blood cells, centrifugation to recover the white blood cells, washing with NaOH and the addition of bovine serum albumin (2, 3, 15-19).

These pre-treatment steps of the blood samples are generally time-consuming, labor-intensive, and can be sample specific. The guanidinium thiocianate method for DNA isolation is not suitable for reliable detection of Mycobacterium tuberculosis in clinical samples. An alternative method of DNA purification with protease K treatment followed by phenol-chloroform extraction has to be employed to relieve the inhibition (20). Separation with a QIAamp kit followed by dialysis with a Millipore filter are required for eliminating the heme inhibition of hepatitis B virus detection (21). In addition, some the above steps carry a risk of target DNA losses and are not suitable for automation. Moreover, even commercial kits specially formulated for DNA purification from blood samples such as QIAmp or GeneReleaser are not always satisfactory. The reason is due to an incomplete removal of Taq inhibitors, which can result in false-negative results. For example, 14% of the human blood samples tested for hepatitis B virus yielded false-negative results when using such blood kits (21).

The objective of achieving specificity of amplification reactions for samples containing whole blood is further complicated by two types of unwanted DNA synthesis reactions that occur during PCR. Both types of side-reactions are frequently competitive with the desired target and can lead to impure product or failed amplification. This is particularly problematic for PCR assays containing a low copy number of the nucleic acid template target, wherein the PCR conditions are modified to include a greater number of amplification cycles to achieve an adequate yield of the desired amplification product.

The first type of unwanted DNA synthesis is priming on less specific sequences in the template. This is only an issue if the template is contaminated with single-stranded nucleic acid or if the template is single-stranded, which is the case if the DNA preparation has been subjected to melting conditions during its isolation.

The second type of unwanted DNA synthesis is primers acting as templates for themselves and/or each other, with at least the result of modifying their 3′ ends by the addition of additional nucleotides. These so-modified primers are able to anneal to the nucleic acid target; however, they do not serve as primers for complementary strand synthesis due to the presence of mismatched nucleotides at the site of elongation between the 3′ end of the primer and the desired target. This problem is often referred to as “primer dimer”, although this name is not accurately descriptive. This problem can often be reduced or avoided by careful primer design, and it is more of a problem with multiplex PCR, since there is more opportunity for accidental homology among multiple pairs of primers.

A procedure known as “hot start PCR” avoids the occurrence of both types of unwanted DNA synthesis side-reactions. According to this method, the enzyme DNA polymerase, or a buffer component essential to its activity, such as the magnesium (II) cation and/or the dNTPs, is withheld from the other PCR assay mixture ingredients until the PCR reaction has been heated to at least the normal primer-annealing (or, preferably, the DNA extension) temperature (55-75° C., optimal 68° C.). At this temperature the primers can presumably not form stable duplexes with themselves or at unwanted template sequences. After the selective temperature is achieved, the omitted component is added to reaction to reconstitute a functional amplification mixture.

Typical hot start PCR procedures are not only labor-intensive, they expose the PCR reactions to contamination with each other and with molecules that have been previously amplified in the thermal cycler machine.

The more standard ways of executing a hot start consist of formulating the PCR reaction in two parts, such that the DNA polymerase is not able to act on the DNA until the two portions are combined at high temperature, usually 65-85° C. For instance, an initial solution containing all of the magnesium is introduced to the reaction tube encapsulated in a wax bead or sealed under a layer of wax. The rest of the reaction, without Mg, is then added, along with an overlay of oil, if appropriate. While the reaction heats for the first cycle, the wax melts and floats to the surface, allowing the magnesium to mix with the reaction volume. The DNA polymerase activity is therefore reconstituted at a temperature that does not allow non-specific or unwanted primer interactions. A great drawback to the wax method comes after the PCR cycling is complete, and the product must be withdrawn for analysis. The wax then tends to plug the pipette tip, greatly adding to the time and effort of reaction analysis.

Recently, a method of hot start which is not hot at all, but which uses anti-Taq antibodies, has been described, patented and made commercially available (33-35). The antibodies largely neutralize the enzyme activity of the Taq polymerase, and can be added any time prior to the primers, or be conveniently present during storage of the stock enzyme. The antibodies are thermolabile, thus permitting the Taq polymerase to resume activity after the first heat step. The antibodies so far developed for this method must be used in 10-fold molar excess and are expensive. Furthermore, the antibodies inhibit some long PCR assays that are conducted with the KlentaqLA polymerase mixture.

A chemically inactivated form of the Taq polymerase has been introduced recently, termed Amplitaq Gold. The nature of the inactivation is proprietary, but the inactivation is reversible by heating the polymerase at 95° C. This method may be even more convenient than the other methods, but it has at least one current disadvantage: the time for reactivation is about 10 minutes at 95° C. This procedure is incompatible with long PCR applications, as this treatment would excessively depurinate nucleic acid targets longer than a few kb.

Thus, the analysis of whole blood samples using PCR would be benefited by the discovery of new reagents and methods that overcome the aforementioned shortcomings of current PCR technologies. The invention disclosed herein addresses and solves many of these shortcomings.

### SUMMARY

In the first aspect, the present invention is a method of obtaining DNA amplification of a nucleic acid target from a volume of whole blood comprising performing DNA amplification in a PCR assay mixture with a blood-resistant polymerase.

In the second aspect, the present invention is a method of obtaining DNA amplification of a nucleic acid target from whole blood that includes the following steps: (1) adding a first solution comprising a DNA amplification cocktail into a reaction vessel; (2) underlayering the first solution with a volume of whole blood to yield a final volume of reaction; and (3) performing a thermal cycling program to effect DNA amplification. The DNA amplification cocktail includes at least one blood-resistant polymerase.

In a third aspect, the present invention is a method of obtaining a hot start for DNA amplification of a nucleic acid target that includes the preparation of the reaction cocktail comprising at least a first volume component and a second volume component. The second volume component is heavier than the first volume component; the first volume component comprises a DNA polymerase cocktail lacking an essential constituent required for DNA amplification activity; the second volume component comprises the essential constituent required for DNA amplification activity; and the second volume component is underlayed below the first volume component without undue mixing before a DNA amplification reaction is initiated.

In a fourth aspect, the present invention is an isolated polypeptide comprising an amino acid sequence comprising at least 80% amino acid sequence identity with KT-1 (SEQ ID NO:2), KT-6 (SEQ ID NO:4), and KT-7 (SEQ ID NO:6), wherein the isolated polypeptide comprises a blood-resistant polymerase.

In a fifth aspect, the present invention is an isolated nucleic acid comprising a nucleotide sequence comprising at least 80% nucleotide sequence identity of Klentaq1 (SEQ ID NO:1), KT-6 (SEQ ID NO:3), and KT-7 (SEQ ID NO:5), wherein the isolated nucleic acid encodes a blood-resistant polymerase.

In a sixth aspect, the present invention is method of screening for a faster elongating polymerase that includes conducting PCR assays on a nucleic acid target using a thermal cycling program, wherein the thermal cycling program includes an extension reaction performed for at most about 20 sec per 2 kilobase pair amplicon and a collection of mutant DNA polymerases to be screened are cold-sensitive.

In a seventh aspect, the present invention is a kit for performing PCR assays on samples of whole blood that includes at least one a blood-resistant polymerase.

Definitions

The term “amplicon” refers to the nucleic acid that is the target of DNA amplification of a PCR assay.

The phrase “amplification activity” refers to the functional ability of a DNA polymerase to synthesize copies of a nucleic acid target under the PCR conditions disclosed herein to yield a quantity of amplified DNA product that is discernable by intercalative dye (e.g., ethidium bromide) staining methods that are well known in the art.

The phrase “homogeneous PCR assay solution” as used herein refers to a solution that is homogenous with respect to the absence of discrete phases. A homogeneous PCR assay solution is one that is typically prepared by mixing the contents of a reaction vessel using a vortexer or comparable mixing apparatus. In the context of heavy hot start PCR assays, the PCR assay solution is composed of two phases prior to initiating the thermal cycling program; that is, the PCR assay solution of a heavy hot start PCR assay is not premixed prior to initiating a thermal cycling program and is not considered a homogenous PCR assay solution.

The phrase “blood-resistant polymerase” as used herein refers to a mutant form of Taq DNA polymerase Klentaq-278 that displays amplification activity in a homogeneous PCR assay solution containing whole blood in the range from about 3% (vol/vol) to about 25% (vol/vol). A mutant form of Klentaq-278 DNA polymerase includes a polypeptide that does not encode the identical amino acid sequence of Klentaq-278 DNA polymerase (SEQ ID NO:2). Examples of such mutant forms include a deletion of at least one amino acid, an insertion of additional amino acids, or a change of at least one amino acid relative to the amino acid sequence of the Klentaq-278 DNA polymerase (SEQ ID NO:2).

The phrase “faster-elongating polymerase” as used herein refers to a derivative of Taq DNA polymerase that displays amplification activity in PCR assays conducted with extension times in the range from about 12 seconds to about 50 seconds to complete up to 2 kb.

The phrase “physiologically compatible buffer” as used herein refers to any solution that is compatible with the function of enzyme activities and enables cells and biological macromolecules to retain their normal physiological and biochemical functions. Typically, a physiologically compatible buffer will include a buffering agent (e.g., TRIS, MES, PO₄, HEPES, etc.), a chelating agent (e.g., EDTA, EGTA, or the like), a salt (e.g., NaCl, KCl, MgCl₂, CaCl₂, NaOAc, KOAc, Mg(OAc)₂, etc.) and optionally a stabilizing agent (e.g., sucrose, glycerine, Tween20, etc.).

The polymerases referred to throughout this description have the following structures and properties: (1) Taq refers to the wild-type, full-length DNA Polymerase from Thermus aquaticus (GenBank Accession No. J04639); also used for chemically modified variants thereof, such as “Amplitaq-Gold;” (2) Klentaq-235 refers to an N-terminal deletion of the first 235 amino acids of Taq. Klentaq-235 is also known in commerce as DeltaTaq, ATaq, Klentaq, and Klentaq5; (3) Klentaq-278 refers to an N-terminal deletion of the first 278 amino acids of Taq (Klentaq-278 is also referred to as “Klentaq1” or “KT-1” or wild-type Klentaq1) and is described in claims 1-5 of U.S. Pat. No. 5,436,149; (4) Klentaq6 (abbreviated as KT-6) refers to Klentaq-278 with 2 amino-acid changes. Klentaq7 (abbreviated as KT-7) refers to Klentaq-278 with 3 amino-acid changes.

The suffix “LA” means “Long and Accurate” and refers to a mixture of thermostable DNA polymerases, after claims 6-16 of U.S. Pat. No. 5,436,149 and Barnes (1994). Major component is usually Taq or Klentaq1. A minor component is usually an archaebacterial DNA polymerase such as Pfu polymerase, Pwu polymerase, Vent polymerase, or Deep Vent polymerase.

KlentaqLA is a mixture of 47:1::Klentaq1:Deep Vent by volume of commercially available enzymes. This mixture also may be modified to 24:1 as noted in the text. Since commercially distributed Klentaq1 is about 15-20 times more concentrated than commercially distributed Deep Vent, the true ratio, by units or protein, is approximately 15-20 times higher, i.e. 705:1 or 360:1

TaqLA is a mixture of 47:1::Taq:Deep Vent, or 16:1::Taq:Pfu, or an unspecified mixture of Taq:Pfu that is commercially known as “TaqPlus.”

Control sequences are DNA sequences that enable the expression of an operably-linked coding sequence in a particular host organism. Prokaryotic control sequences include promoters, operator sequences, and ribosome binding sites. Eukaryotic cells utilize promoters, polyadenylation signals, and enhancers.

The phrase “operably-linked” refers to a nucleic acid when it is placed into a functional relationship with another nucleic acid sequence. For example, a promoter or enhancer is operably-linked to a coding sequence if it affects the transcription of the sequence, or a ribosome-binding site is operably-linked to a coding sequence if positioned to facilitate translation. Generally, “operably-linked” means that the DNA sequences being linked are contiguous. However, enhancers do not have to be contiguous. Linking is accomplished by conventional recombinant DNA methods.

The phrase “a reaction vessel” refers to any container that may used for performing a biological, biochemical or chemical reaction. In the context of PCR assays, a reaction vessel is any suitable container that can withstand the temperatures carried during a typical DNA amplification reaction. Preferably, a reaction vessel that used for PCR assays includes a tube fitted with a closure, wherein both the tube and the closure are made of polymeric material such as polypropylene or similar material commonly employed in the art.

The phrase “isolated nucleic acid molecule” is purified from the setting in which it is found in nature and is separated from at least one contaminant nucleic acid molecule.

The phrase “isolated polypeptide molecule” is purified from the setting in which it is found in nature and is separated from at least one contaminant polypeptide molecule.

The phrase “purified polypeptide” refers to a polypeptide molecule that has been purified to greater than 80% homogeneity by SDS-PAGE under non-reducing or reducing conditions using Coomassie blue or silver stain. Isolated polypeptides include those expressed heterologously in genetically engineered cells or expressed in vitro. Ordinarily, isolated polypeptides are prepared by at least one purification step.

### BRIEF DESCRIPTION OF THE DRAWINGS

FIG. 1 depicts results of PCR assays with different forms of Klentaq polymerase (38 mutants and wild-type form of Klentaq) conducted in the presence of inhibitory amounts of blood. Clones KT-6 and KT-7 were capable of amplifying a 1.65 kbp target DNA from added plasmid template in the presence of 10% whole human blood;

FIG. 2A depicts results of PCR amplification of a 1.65 kbp target DNA from an exogenous plasmid template with two mutant forms of Klentaq (KT-6 and KT-7), wild-type Klentaq1 (KT-1) and wild-type Taq (Taq) in the presence of increasing amounts of whole human blood (lane 1: 0%; lane 2: 10%; lane 3: 15%);

FIG. 2B depicts results of PCR amplification of a 0.32 kbp target DNA directly from whole blood with two mutant forms of Klentaq (KT-6 and KT-7) in the presence of increasing amounts of whole human blood (lane 1: 0%; lane 2: 5%; lane 3: 10%; lane 4: 15%);

FIG. 3A depicts results of PCR amplification of a 1.65 kbp target DNA as a function of elongation time for reactions containing two mutant Klentaq polymerases (KT-6 (lane 2) and KT-7 (lane 3)) Klentaq1 polymerase (lane 1) and another commercially available Taq polymerase (lane 4). The extension times are indicated below the panel;

FIG. 3B depicts results of PCR amplification of a 1.65 kbp target DNA as a function of exogenous template concentration and elongation time for reactions containing a mutant Klentaq polymerase (KT-7) and a DNA polymerase that possesses the highest prior art elongation rates (Z-TAQ). The added nucleic acid target amounts are as follows: 0.5 ng (lane 1); 0.25 ng (lane 2); 0.125 ng (lane 3); and 0.06 ng (lane 4). The extension times are as follows: 60 sec (upper panel); 15 sec (central panel); and 12 sec (lower panel);

FIG. 4A depicts results of PCR amplification of a 1.65 kbp target DNA from oil:water emulsions containing bacterial cells expressing wild-type Klentaq in the presence (lanes 1 and 2) or absence (lanes 3 and 4) of 10% whole human blood and using KT1 (SEQ ID NO:11) and RevTaqH (SEQ ID NO:12), which result in the specific amplification of a 1.65 kbp target fragment;

FIG. 4B depicts results of PCR amplification of a 1.65 kbp target DNA from oil:water emulsions containing bacterial cells expressing either the wild-type Klentaq polymerase (lanes 2 and 4) or a library of mutagenized Klentaq clones (lanes 1 and 3) in the presence (lanes 1 and 2) or absence (lanes 3 and 4) of 10% whole human blood and using KT1 (SEQ ID NO:11) and RevTaqH (SEQ ID NO:12), which result in the specific amplification of a 1.65 kbp target fragment;

FIG. 5A illustrates the results of heavy hot start PCR assays (100 μL reaction volumes) conducted with KT-1 (SEQ ID NO:2), KT-6 (SEQ ID NO:4) and KT-7 (SEQ ID NO:6) in the presence of whole blood and under different conditions of pre-treatment of the reaction samples prior to initiating the thermal cycling program. The asterisks indicate those reaction vessels wherein the heavy and light volume component layers were premixed by vortexing, i.e., reactions that a contain homogeneous PCR assay solution and that were not subjected to a heavy hot start procedure as described herein. Lanes 1-13, 15 and 17 are PCR assays directed toward the amplification of a 1.1 kbp target from the human CCR5 gene whereas lanes 14, 16, and 18 are PCR assays directed toward the amplification of a 2.5 kbp target from the human CCR5 gene;

FIG. 5B illustrates an example of PCR assay tubes from reaction mixtures 9-14 of FIG. 5A that were not mixed prior to initiating the thermal cycling reaction (heavy hot start reactions; reaction nos. 9, 10, 13, and 14) or mixed by vortexing briefly prior to initiating the thermal cycling reaction (non-heavy hot start reactions; reaction nos. 11 and 12);

FIG. 6 depicts results of heavy hot start PCR amplification of a 0.5 kbp target from the human CCR5 gene of cells present in whole blood. The reactions were conducted in the presence of whole blood in the lower layer at the percentages indicated below each lane (vol/vol; adjusted for total volume of both layers), in the absence of whole blood (indicated by “0”), or in absence of blood and in the presence of 10 ng of human DNA (indicated by “0+”);

FIG. 7 depicts results of PCR assays directed toward the amplification of a 2.5 kbp target from the human CCR5 gene derived from 2 ng of genomic DNA (designated “DNA”) or from 3% whole blood (vol/vol) (designated “Blood”) using either KT-1 (SEQ ID NO:2), KT-6 (SEQ ID NO:4), or KT-7 (SEQ ID NO:7) in the absence of Deep Vent polymerase (lanes 1-3 and 7-9) or in the presence of Deep Vent polymerase (lanes 4-6 and 10-12), wherein the ratio of the KT enzyme to Deep Vent polymerase is about 360 to 1.

### DETAILED DESCRIPTION

The present invention makes use of the discovery that Taq polymerases bearing certain N-terminal deletions are unusually resistant to whole blood, making them ideally suited for use in analytical PCR assays of nucleic acid targets from human blood. Mutant(s) of Taq DNA polymerase with even higher resistance to blood inhibitors have been developed that remain fully functional in the presence of at least 20-25% blood or the equivalent of blood fractions. This level of blood tolerance exceeds that of the existing thermostable DNA polymerases (and even exceeds the amount of blood that can be practically or conveniently handled in the PCR analysis due to physical clumping). Moreover, mutants that display a high resistance to blood inhibitors have been identified that possess faster elongation rates. The use of these novel enzymes is expected to simplify and accelerate the performance of clinical and forensic tests as well as render such tests more sensitive and economical. Finally, the present invention provides methods for enhancing DNA amplification specificity using these polymerases with samples from whole blood. These Taq polymerase mutants and methods for their use are described below.

Identification of Klentaq Mutants that are Highly Resistant to Blood Inhibition.

Klentaq1 polymerase (SEQ ID NO: 1 (nucleic acid) and SEQ ID NO:2 (polypeptide)) is an improved and more robust version of the Taq polymerase that bears an N-terminal deletion of 278 amino acids from the full-length (832 amino acids) enzyme. Klentaq 1 displays higher fidelity and greater thermostability than Taq. Klentaq1 is also inhibited to a lesser extent than Taq when the polymerase is used in PCR assays carried out in the presence of blood products. For example, the purified Klentaq1 enzyme easily amplifies a nucleic acid target in the presence of about 5% whole blood in reaction mixture (vol/vol). This was a highly unexpected result, as the full-length Taq enzyme is completely inhibited in a blood concentration range of about 0.004% to about 0.2% whole blood in the reaction mixture (vol/vol). No correlation between the N-terminal deletion of Taq, which generates Klentaq 1, and the blood resistance feature of the enzyme has been reported.

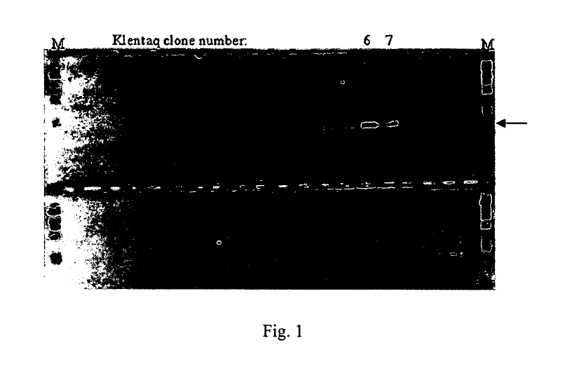

Several mutant Klentaq clones were analyzed by PCR assays for their ability to tolerate whole blood. About 40 mutagenized, yet PCR-functional Klentaq clones were constructed and tested in PCR assay mixtures containing about 10% whole human blood (vol/vol). These 40 clones are cold-sensitive or are mutants of clones whose enzyme product exhibited the cold-sensitive phenotype. The cold-sensitivity of the additionally mutant clones has not yet been determined. Remarkably, two mutants of this small collection, KT-6 (SEQ ID NO: 3 (nucleic acid); SEQ ID NO: 4 (polypeptide)) and KT-7 (SEQ ID: 5 (nucleic acid); SEQ ID NO:6 (polypeptide)), clearly outperformed the rest of the clones and the wild-type Klentaq1 protein under these conditions (FIG. 1).

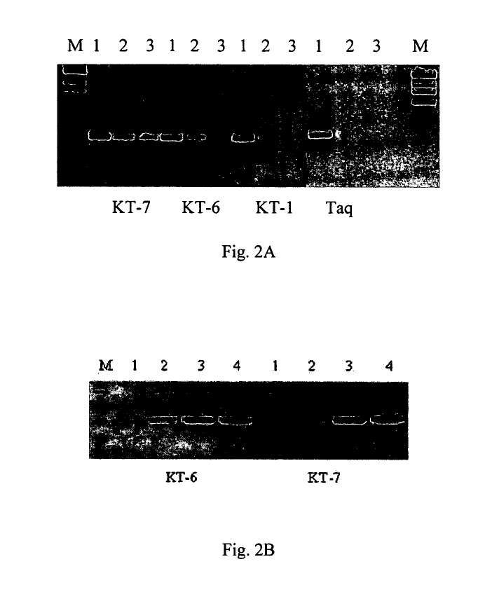

These results were confirmed by performing PCR assays in the presence of increasing amounts of whole blood. As shown in FIG. 2A, clones KT-6 and KT-7 remained functionally active in the presence of at least about 10% whole blood (vol/vol). The presence of these blood concentrations in PCR assays were inhibitory for Klentaq and Taq (Roche) (see FIG. 2A and Example 406). Moreover, KT-6 and KT-7 were able to amplify an endogenous gene target directly from blood cells present in reactions containing 15% whole human blood without any DNA purification step (FIG. 2 B).

The foregoing results reveal that whole blood may be used directly in screening assays to identify mutants of Klentaq-278 that are even more resistant to blood. The present invention is drawn in part to mutant forms of the Klentaq 1 polymerase that display activity in PCR assays containing from about 5% whole blood to about 25% whole blood in the reaction mixture (vol/vol). More preferably, the invention is drawn to mutant forms of the Klentaq polymerase that display amplification activity in PCR assays containing from about 5% whole blood to about 20% whole blood in the reaction mixture (vol/vol), including 6%, 7%, 8%, 9%, 10%, 11%, 12%, 13%, 14%, 15%, 16%, 17%, 18%, and 19% whole blood in the reaction mixture (vol/vol).

Identification of Klentaq Mutants with Faster DNA Elongation Rates

A “rapid” thermostable DNA polymerase mutant has been discovered that displays a faster DNA elongation rate than found for the wild-type Klentaq-278 polymerase. By lowering the DNA extension times during PCR, certain PCR conditions have been determined where the elongation step in the cycle becomes limiting for successful amplification by the wild-type Klentaq-278 enzyme. In the case of using the Klentaq-278 gene as a target (1.65 kb long), the minimum extension time required was about 1 minute. For example, the Klentaq-278 polymerase did not possess amplification activity in PCR assays performed under conditions that employ extension times of 50 seconds. Similar results were obtained with Taq enzyme.

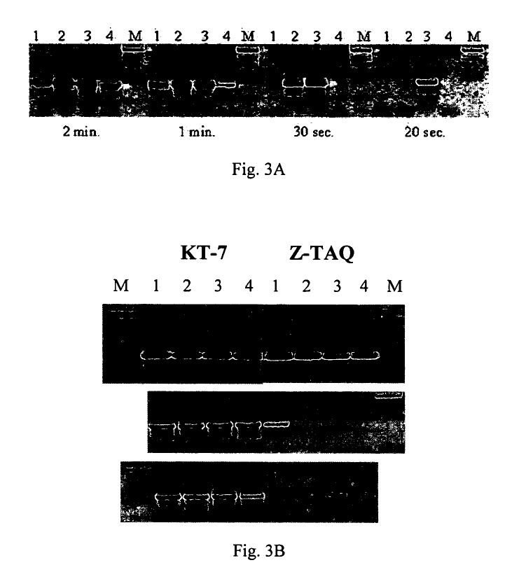

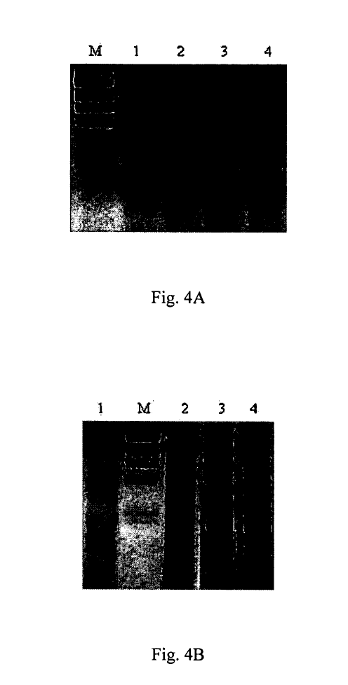

About 40 functional mutant Klentaq clones were evaluated as a function of elongation rate. A 30 sec extension time was initially employed in the PCR assays, which reflect conditions that were found ineffective for the wild-type Klentaq and AmpliTaq Gold. Interestingly, the mutants KT-6 (SEQ ID NO:4) and KT-7 (SEQ ID NO:6) were able to efficiently amplify the target with this shorter extension time (see FIG. 3). This feature of the two mutant enzymes was confirmed in further tests, wherein one of them (clone KT-7 (SEQ ID NO:6)) yielded amplification products with a 20 sec extension time (FIG. 3 A). This enzyme feature was characterized further and yielded good amplification products even with 15 and 12 sec extension times. Remarkably, the selected mutant completely outperformed the Z-TAQ (Tamara) at these low extension times (FIG. 3 B, central and lower panels). This is noteworthy because Z-TAQ is one of the fastest DNA elongating PCR enzymes that is commercially available.

These results demonstrate that the elongation speed of Klentaq DNA polymerase enzyme can be improved by mutagenesis (5-6 fold faster in this first identified mutant clone). The present invention is drawn in part to mutant forms of the Klentaq polymerase that display increased elongation rate in PCR assays under conditions where the wild-type Klentaq-278 enzyme fails to display successful amplification activity. Preferably, the invention is drawn to mutant forms of the Klentaq-278 polymerase that display amplification activity in PCR assays under conditions where the elongation step is time-limiting for the reaction with the wild-type Klentaq-278 polymerase. Even more preferably, the invention is drawn to mutant forms of Klentaq-278 polymerase that display amplification activity under PCR conditions disclosed herein and having extension times in the range from about 12 sec to about 50 sec, including 15 sec, 18 sec, 20 sec, 22 sec, 24 sec, 25 sec, 26 sec, 28 sec, 30 sec, 32 sec, 34 sec, 36 sec, 38 sec, 40 sec, 42 sec, 44 sec, 45 sec, 46 sec, and 48 sec.

Heavy Hot Start PCR Procedures and Applications to Whole Blood PCR

The new protocol described here uses no wax or antibodies, and requires no manipulations once the thermal cycling program has commenced. This protocol uses two aqueous layers at the time of setup of the PCR assay. The lower layer, which represents about 1/10 to about ¼ of the final volume, includes the dNTPs and magnesium(II) that is required for the reaction. The upper layer contains the polymerase enzyme, the primers, and the nucleic acid target. Both layers contain equivalent concentrations of other buffer components at the concentrations required for amplification. The lower layer also contains a constituent to make it heavy, such as about 10-20% (wt/vol) sucrose, sorbitol or DMSO (or a suitable combination of similar reagents compatible with PCR up to about 10-20% (wt/vol)).

Optionally, other components that impart greater density to the lower layer may substitute for or supplement the items described above. For instance, Baskaran and co-workers have demonstrated that 1.4 M betaine, 5% DMSO is good for PCR assays involving nucleic acid targets possessing high GC content (36). These results suggest that inclusion of 2.8 M betaine, 10% DMSO is feasible as the heavy start component of the lower layer containing the MgCl₂ and the dNTPs. Optionally and routinely, color in the form of 0.05% cresol red is also included in the lower, heavy layer.

In reactions that include whole blood, the addition of components that impart greater density to the lower layer and a color agent are not required. These features are superfluous because whole blood imparts a density to the lower layer that approximates that of the aforementioned heavy layer components and because the hemoglobin of blood provides color. In reactions containing whole blood, the template is included in the heavy layer, and all other components of the reaction are in the upper layer. The range of volumes appropriate to the use of whole blood in the heavy layer comprises 1% to 25%.

Some adverse components of blood attack various components of the PCR reaction, such as the enzyme or the primers, yet the adverse components may be heat labile. Thus, our addition of the blood carefully as an unmixed underlay allows it to be added without significant contact with the putatively sensitive PCR reaction components. Upon heating to normal PCR thermal cycling temperatures of 90-95 degrees C., we have observed that many of the blood components are denatured and aggregated in place, are visible as brown after the cycling, and either did not mix with the PCR components before being inactivated by the heat, or never did mix appreciably with the PCR reaction components. Nevertheless, we report that the genomic DNA template, and presumably other target templates such as viral and other microbial genomes, become timely available to the amplification reaction by convective mixing.

This principal of segregating heat labile inhibitors during reaction setup may have application to other situations of complex or environmental samples that do not involve blood.

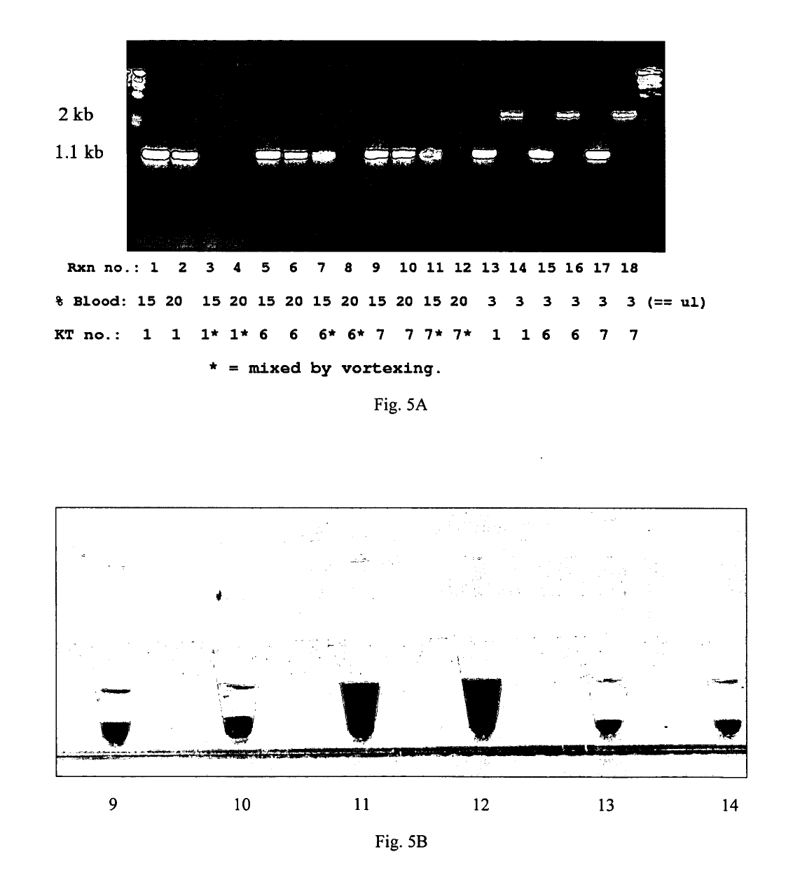

Preferably the lower, heavy layer of each PCR reaction is added last, and importantly, without mixing. In the preferred embodiment, mixing of the layers occurs by diffusion and/or convection after the thermal cycler has warmed and cooled the reaction to begin the PCR process. Layered reaction tubes containing whole blood that are experimentally premixed by vortex treatment are variably unable to support PCR amplification activity, depending on the resistance of the reaction components, and we have discovered that the most sensitive component is the DNA polymerase enzyme (FIG. 5A). FIG. 5B illustrates an example of PCR assay tubes that contain discrete layers prior to reaction and the mixing of the layers during reaction.

It is well understood to one of ordinary skill in the art that the combinations of components in the separate layers may be formulated in a variety of permutations. The only criteria that must be met in the present invention is that the polymerase is separated from at least one component essential to the amplification reaction (e.g., the primers, and/or the template, and/or Mg²⁺), that the lower layer contains a component that imparts greater density to the solution, and that the mixing of the two layers results in reconstitution of the PCR assay conditions to permit amplification activity.

Because the inclusion of heavy reagents, such as sucrose, sorbitol or DMSO will decrease slightly the melting temperature of the nucleic acid target, the denaturation step of the PCR cycle may have to be reduced by about 1-2° C. to compensate for this effect.

Mutant forms of KLENTAQ-278 used in the invention includes the amino acid sequence (SEQ ID NO:2) encoded by the nucleic acid (SEQ ID NO:1) whose sequences comprise the sequences provided in Tables 1-6, or other codons that encode those amino acids, or those amino acids with a few extra codons on the amino terminus thereof. The invention also uses a mutant or variant gene encoding KLENTAQ-278, any of whose bases may be changed from the corresponding base shown in Tables 1-6 while still encoding a protein that maintains the activities and physiological functions of KLENTAQ-278, or a slightly longer or shorter protein at the N-terminus. Further included are nucleic acids whose sequences are complementary to those just described, including complementary nucleic acid fragments. Additionally, nucleic acids or nucleic acid fragments, or complements thereto, whose structures include chemical modifications, are also included. Such modifications include, by way of nonlimiting example, modified bases, and nucleic acids whose sugar phosphate backbones are modified or derivatized. In the mutant or variant nucleic acids, and their complements, up to 20% or more of the bases may be so changed.

The invention also includes the use of polypeptides and nucleotides having 80-100% sequence identity to SEQ ID NOS:1-6, including 81, 82, 83, 84, 85, 86, 87, 88, 89, 90, 91, 92, 93, 94, 95, 96, 97, 98 and 99%, sequence identity to SEQ ID NOS:1-8, as well as nucleotides encoding any of these polypeptides, and complements of any of these nucleotides. In the case of Klentaq1 (SEQ ID NO:1), the invention includes mutant forms that contain at least 2 codon changes in the open reading frame of Klentaq1 (SEQ ID NO:2). Examples of such mutations include DNA polymerases of KT-6 (SEQ ID NO:4) and KT-7 (SEQ ID NO:6).

“Percent (%) nucleic acid sequence identity” with respect to KLENTAQ-278-encoding nucleic acid sequences identified herein is defined as the percentage of nucleotides in a candidate sequence that are identical with the nucleotides in the KLENTAQ-278 sequence of interest, after aligning the sequences and introducing gaps, if necessary, to achieve the maximum percent sequence identity. Alignment for purposes of determining % nucleic acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

Percentage Sequence Identity

When nucleotide sequences are aligned, the percent (%) nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) can be calculated as follows:
% nucleic acid sequence identity=W/Z·100
where
W is the number of nucleotides scored as identical matches by the sequence alignment program's or algorithm's alignment of C and D and
Z is the total number of nucleotides in D.

When the length of nucleic acid sequence C is not equal to the length of nucleic acid sequence D, the % nucleic acid sequence identity of C to D will not equal the % nucleic acid sequence identity of D to C.

“Percent (%) amino acid sequence identity” is defined as the percentage of amino acid residues that are identical with amino acid residues in the disclosed KLENTAQ-278 polypeptide sequence in a candidate sequence when the two sequences are aligned. To determine % amino acid identity, sequences are aligned and if necessary, gaps are introduced to achieve the maximum % sequence identity; conservative substitutions are not considered as part of the sequence identity. Amino acid sequence alignment procedures to determine percent identity are well known to those of skill in the art. Often publicly available computer software such as BLAST, BLAST2, ALIGN2 or Megalign (DNASTAR) software is used to align peptide sequences. Those skilled in the art can determine appropriate parameters for measuring alignment, including any algorithms needed to achieve maximal alignment over the full length of the sequences being compared.

When amino acid sequences are aligned, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) can be calculated as:
% amino acid sequence identity=X/Y·100
where
X is the number of amino acid residues scored as identical matches by the sequence alignment program's or algorithm's alignment of A and B and
Y is the total number of amino acid residues in B.

If the length of amino acid sequence A is not equal to the length of amino acid sequence B, the % amino acid sequence identity of A to B will not equal the % amino acid sequence identity of B to A.

A nucleic acid molecule used in the invention, e.g., a nucleic acid molecule having the nucleotide sequence of SEQ ID NOS:1, 3, or 5, or a complement of this aforementioned nucleotide sequence, can be isolated using standard molecular biology techniques and the provided sequence information. Using all or a portion of the nucleic acid sequence of SEQ ID NOS:1, 3, or 5 as a hybridization probe, KLENTAQ-278 gene molecules can be isolated using standard hybridization and cloning techniques (29, 30).

PCR amplification techniques can be used to amplify KLENTAQ-278-encoding DNA using Thermus aquaticus genomic DNA as a template and appropriate oligonucleotide primers. Furthermore, oligonucleotides corresponding to KLENTAQ-278 gene sequences can be prepared by standard synthetic techniques, e.g., an automated DNA synthesizer.

Klentaq-278 is the subject of U.S. Pat. No. 5,436,149 (31), which is incorporated herein by reference.

Klentaq-235 is the subject of U.S. Pat. No. 5,616,494 (32), which is incorporated herein by reference.

Medical Applications

The applications of the present invention include diagnostic evaluations of whole blood samples for the presence and status of genetic disorders (e.g., cancer, blood disorders, diabetes, etc.) and diseases caused by blood borne microbial agents (e.g., viruses, bacteria, fungi, etc.); tissue-typing using polymorphisms, and forensic research. One of ordinary skill would recognize the utilities of blood-resistant polymerases and high elongating polymerases of the present invention toward advancing the application of PCR to whole blood samples directed to these objectives.

Kits

The present invention also contemplates kits that may be employed in the clinical setting or in the field for permitting a simplified set of reagents for rapid PCR analysis of whole blood samples using the blood-resistant polymerases and high elongating polymerases of the present invention. Kits would typically include suitable oligonucleotide primers, PCR reaction buffer components, control solutions, and the blood-resistant polymerase, as well as instructions for the kit's use.

### EXAMPLES

### Example 1

### Screening of Mutagenized Klentaq Clones for Blood-Resistant Mutant Enzyme Activity

In order to functionally characterize new mutants, it is desirable to produce highly-purified enzyme from expression systems. The procedure, including PEI treatment, BioRex-70 and Heparin-Agarose chromatography, yielded DNA-free and nuclease-free Klentaq enzyme purified to homogeneity, as judged by a single band in Coomassie stained protein gel (23). The same purification procedure also worked very well for purification of cold-sensitive Klentaq mutants (23). This procedure is readily adaptable to accommodate purification of mutant polymerases that display unusual features such as changed affinity and elution profile on a particular chromatography resin. The efficiency of each step in the purification scheme is monitored easily by a standard DNA incorporation assay.

The amplification activity of the obtained mutant enzymes were extensively evaluated in PCR amplification of various gene targets. The new enzymes were tested both in conventional and real-time PCR with SYBR green fluorescent detection. These tests included at least about 20% whole human blood (untreated, EDTA-treated or heparinized), or blood IgG and hemoglobin fractions equivalent. Optionally, the differential sensitivities that the polymerase mutants display toward whole blood were evaluated by performing an amplification activity titration experiment with increasing incremental amounts of whole blood added to the assay mixtures from about 5% whole blood (vol/vol) to about 25% whole blood (vol/vol).

FIG. 1 illustrates the results of typical PCR assays with homogeneous PCR assay solutions containing 10% whole blood (vol/vol), wherein the Klentaq gene represent the target nucleic acid. The primers used in the PCR assays comprise KT1 (SEQ ID NO:11) and RevTaqH (SEQ ID NO:12), which result in the specific amplification of a 1.65 kbp target fragment.

FIG. 2A illustrates the results of typical PCR assays with homogeneous PCR assay solutions containing different amounts of whole blood (vol/vol) in the reaction, wherein the Klentaq gene represents the target nucleic acid. The primers used in the PCR assays comprise KT1 (SEQ ID NO:11) and RevTaqH (SEQ ID NO:12), which result in the specific amplification of a 1.65 kbp target fragment. FIG. 2B illustrates the results of typical PCR assays with homogeneous PCR assay solutions containing different amounts of whole blood (vol/vol) in the reaction, wherein the endogenous human gene from blood represents the target nucleic acid. The primers used in the PCR assays comprise DMDex21f (SEQ ID NO:13) and DMDex21r (SEQ ID NO:14), which result in the specific amplification of a 0.32 kbp target fragment of the endogenous human dystrophy gene.

### Example 2

### Screening of Mutagenized Klentaq for Mutants with Faster DNA Elongation Rate

The screening factor here will be simply shortening the DNA extension step of the PCR cycle beyond the point where the wild-type or prior art enzyme stops working. As shown in FIG. 3, in the case when wild-type Klentaq amplifies its own gene the amplification efficiency was significantly lower at 60 seconds extension step. Additional tests with discrete extension times showed that the Klentaq polymerase did not display amplification activity in PCR assays performed under conditions that employ an extension time of about 50 sec or less. On the other hand, mutant Klentaq clone KT-7 displayed amplification activity with the same target in PCR assays under conditions having an extension step of as little as about 12 sec. For the evaluation of fast-elongating mutants, extension times in the PCR cycle not exceeding 20 sec. per 2 kb amplicon were used. For these experiments, the PCR assays were conducted using homogeneous PCR assay solutions with KT 1 (SEQ ID NO:11) and RevTaqH (SEQ ID NO:12), which result in the specific amplification of a 1.65 kbp target fragment.

### Example 3

### Hot Start Achieved by Underlay of Heavy Liquid Component can Enhance Yield of Specific Amplification Products—“Heavy Hot Start” Amplification

This amplification procedure permits one to obtain an enhanced specificity and reliability from a PCR assay. The strategy is also amenable to PCR assays involving whole blood, as described below. In two preferred embodiments, two heavy hot start mixes are disclosed that differ mainly in the amounts of Mg²⁺ and dNTPs present in the reaction mixture, since the optimum Mg²⁺ and dNTP concentrations for Klentaq1 and KlentaqLA is higher than for Taq and TaqLA. These heavy hot start mixes can be stored for at least a month at 4° C.

10×TCA is 500 mM Tris-HCl pH 9.2, 160 mM ammonium sulfate. When the pH of the Tris-HCl stock was adjusted to pH 9.2, the pH of the aliquots was measured at a buffer concentration of 50 mM in water at room temperature. The concentration of the 1 M MgCl₂ stock was confirmed by determining the refractive index of the solution using a refractometer and by reference to Refractive Index-Concentration Data in a technical manual, such as THE HANDBOOK OF CHEMISTRY AND PHYSICS by Chemical Rubber Company.

The heavy mix recipe for the KlentaqLA yielded a final Mg(II) cation concentration that was 2.5 mM greater than the total concentration of the dNTP. This heavy mix recipe consists of the following components: 100 μl of 10×TCA; 100 μl of a dNTP mix consisting of 10 mM dATP, 10 mM dGTP, 10 mM dCTP, and 10 mM dTTP; 140 μl of 100 mM MgCl₂, 67 μl of 0.75 mM Cresol Red, 4.25 mM Tris Base, 400 μl of 50% Sucrose or Sorbitol; and 193 μl of water to 1 ml.

The heavy mix recipe for Taq or TaqLA yielded final Mg(II) cation concentration that was 0.75 mM greater than the total concentration of the dNTPs. This heavy mix recipe consists of the following components: 100 μl of 10×TCA; 94 μl of 100 mM MgCl₂, 16 μl of 100 mM dATP; 16 μl of 100 mM dGTP; 16 μl of 100 mM dCTP; 16 μl of 100 mM dTTP; 67 μl of 0.75 mM Cresol Red, 4.25 mM Tris Base 400 μl of 50% Sucrose or Sorbitol; and 275 μl of water to 1 ml.

Typical reaction mixtures were assembled with the following components: 3.75 μl 10×TCA; 1.0 ng target DNA; 1.0 μl (each) 10 μM primers; 0.25 to 0.50 μl enzyme; 30.25 μl water to a final volume of 37.5 μl. This initial mixture represented the top layer. The top layer was added to the PCR assay tube, followed by the addition of oil (if desired or necessary). The PCR tube was subjected to a brief centrifugation step to resolve the aqueous and oil layers. Finally, 13.0 μl of heavy mix was added as an underlayer of the PCR tube contents without mixing. The tubes were closed and carefully carried to and installed into the thermal cycler without undue agitation. The thermal cycler was set to start with a 5 min heating step from 60° C. to 68° C. before the first heat denaturation step. A visual inspection of the tubes thereafter confirmed that the two layers had already mixed during this time.

For heavy hot start PCR assays that included whole blood in the heavy layer, the following experiment was performed. One hundred microliter reactions were assembled with the whole blood being added last. The top layer consisted of 80 μl mixtures, wherein each mixture contained 0.25 μl of polymerase selected from the group consisting of Klentaq1 (Klentaq-278), Klentaq5 (Klentaq-235), Klentaq6, Klentaq7, additional mutants, and Taq. Before the blood was added, water was added to complement the blood volume, so that at the final volume would be 100 μl, even though the volume of the heavy, whole blood underlay ranged from 0.5 μl to 20 μl. The blood was carefully added at the bottom of the tubes, underneath the 80 μl top layer. For example, in PCR assays that contained 0.5 μl of blood, 19.5 μl of water was added to the upper layer before the blood was added as an underlay at the bottom of the tube. The layers were not manually mixed before the PCR assay was performed. The primers were present at 20 pmoles each per 100 μl reaction. The buffer was KLA pH 9, the concentration of dNTPs was 100 μM each, and 1.3 M betaine was present (all concentrations as final in the 100 μl). Ten nanograms of human DNA (from Novagen) was included in the two of the no-blood reactions (the ones catalyzed by Klentaq-235 and Taq) (indicated by lanes denoted by “0+”) to provide a positive control for the polymerase activity. The thermal cycling program was 3 min preheat at 60° C., 35 cycles of (71 sec at 93° C., 60 sec at 60° C., and 5 min at 68° C.).

FIG. 5A illustrates the results of heavy hot start PCR assays (100 μl reaction volumes) conducted with KT-1 (SEQ ID NO:2), KT-6 (SEQ ID NO:4) and KT-7 (SEQ ID NO:6) in the presence of whole blood and under different conditions of pre-treatment of the reaction samples prior to initiating the thermal cycling program. The asterisks indicate those reaction vessels wherein the heavy and light volume component layers were premixed by vortexing, i.e., reactions that a contain homogeneous PCR assay solution and that were not subjected to a heavy hot start procedure as described herein. Lanes 1-13, 15 and 17 are PCR assays directed toward the amplification of a 1.1 kbp target from the human CCR5 gene using ccr5+1 kb (SEQ ID NO:9) and CCR5-KOZ (SEQ ID NO:7). Lanes 14, 16, and 18 are PCR assays directed toward the amplification of a 2.5 kbp target from the human CCR5 gene using CCR5-2 kb (SEQ ID NO:8) and ccr5deltaRT (SEQ ID NO:10).

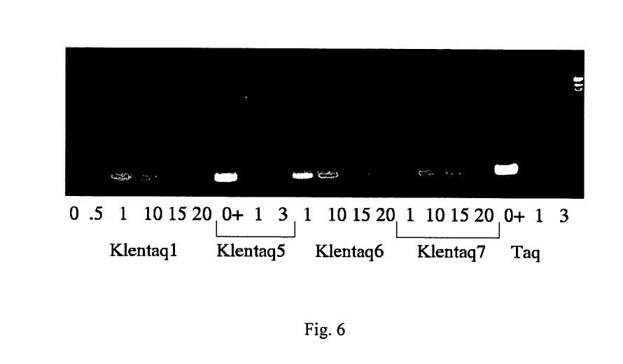

FIG. 6 depicts the additional results of this type of experiment. The amplification activity was revealed by the specific amplification of a 0.5 kbp DNA product from CCR5 gene endogenous to the human cells in the blood (except for the lanes indicated by “0+”. KT-1 (SEQ ID NO:2), KT-6 (SEQ ID NO:4), and KT-7 (SEQ ID NO:6) displayed DNA amplification activity in reaction containing from about 1% whole blood (vol/vol) to about 20% whole blood (vol/vol) whereas Klentaq5 and Taq did not display amplification activity in reactions containing as little as about 1% whole blood (vol/vol). The primers used to generate this amplification product were CCR5-D5 (SEQ ID NO:15) and CCR5-D3 (SEQ ID NO:16).

### Example 4

### Whole Blood PCR Assays that Employ KT Mutant Polymerases with a Second Thermostable DNA Polymerase Having a 3′-Exonuclease Activity

This example shows that long and accurate PCR works with whole blood as the template. Since long and accurate PCR (U.S. Pat. No. 5,436,149, claims 6-16) comprises the use of a mixture of DNA polymerases, this example also illustrates that the minor component of the mixture, an archaebacterial DNA polymerase which is thermostable and which exhibits 3′-exonuclease activity, is surprisingly active with whole blood.

The master PCR cocktail was assembled as follows:
200 μl 10×KLA pH 9
20 μl 10/40 (mix of 10 mM each dNTP and 40 mM MgCl₂)
520 μl 5 M Betaine
40 μl primer CCR5-2 kb (SEQ ID NO:8)
40 μl primer ccr5deltaRT (SEQ ID NO:10)
1120 μl water to make 20×97 μl reaction mixture aliquots
1940 μl total cocktail volume

It is worth noting that the PCR cocktail lacked target nucleic acid template and the DNA polymerase at this stage.

Enzyme dilutions were prepared on ice by mixing them with a portion of the master mix as follows: six aliquots (75 μl each) of master mix were withdrawn and added to an aliquot (0.75 μl) of enzymes KT-1 (SEQ ID NO:2), KT-6 (SEQ ID NO:4), or KT-7 (SEQ ID NO:6) each at about 30 U/11, and the same three enzymes that have been previously mixed with 1:24 dilution volume of the archaebacterial enzyme Deep Vent, which is available commercially at 2 U/μl. These latter enzyme mixtures possessed a ratio of KT enzyme to Deep Vent enzyme of about 1:360.

Aliquots of the master mix (72 μl) were dispensed to reaction tubes, then aliquots of the appropriate enzyme dilution mix (25 μl) were dispensed into the reaction tubes to provide for a total volume of 97 μl.

Pure human DNA (Novagen), stored at a temperature of 4° C. and at a concentration of 3 ng/μl, was diluted 3-fold with standard TEN buffer (10 mM Tris pH 7.9, 10 mM NaCl, 0.1 mM EDTA) to make 1 ng/μl, and then an aliquot of this solution (3 μl) was pipetted into the aforementioned 97 μl mixture to yield the final PCR assay mastermix.

Whole blood, which is typically stored in an aliquot of 0.5 ml with 4.5 mM EDTA at −80° C., was thawed at room temperature for about 15 to 30 minutes and mixed by gentle inversion before 3 μl was pipetted underneath the aforementioned 97 μl mixture in additional PCR reaction tubes, avoiding mixing. The pipettor was set to 3.2 μl, and care was exercised not eject the last small amount of blood volume (˜0.2 μl), so as to avoid injecting a bubble of air into the PCR assay solution and thereby disturb the heavy phase at the bottom of the tube.

Thermal cycling for the PCR amplification was carried out using a similar program as described above (2 minutes at 93 degrees C., followed by 33 cycles of (71 seconds 93 degrees, 1 minute 60 degrees, 10 minutes 68 degrees). After the PCR assays were completed, aliquots of the reactions (18 μl) were mixed with 4.4 μl of blue dye mix, and analyzed by electrophoresis on a 1.4% agarose gel.

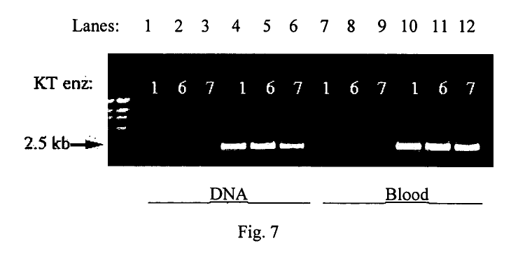

FIG. 7 illustrates that little or no PCR product of the expected size (2.5 kbp) is obtained unless an amount of Deep Vent polymerase is present to complement the major DNA polymerase Klentaq1 (SEQ ID NO:2), Klentaq6 (SEQ ID NO:4), or Klentaq7 (SEQ ID NO:6), all of which perform equally well under these conditions.

Sequence Information

The nucleic acids and polypeptides of the various DNA polymerases described in this application include the sequences shown in Tables 1-6. The oligonucleotide primers described in this application include the sequences shown in Table 7.

TABLE 1
Nucleic acid sequence of Klentaq-278 (SEQ ID NO:1)¹

```
atgggg
1
ctcctccacg agttcggcct tctggaaagc cccaaggccc tggaggaggc cccctggccc
      10 20 30 40 50 60
ccgccggaag gggccttcgt gggctttgtg ctttcccgca aggagcccat gtgggccgat
      70 80 90 100 110 120
cttctggccc tggccgccgc cagggggggc cgggtccacc gggcccccga gccttataaa
      130 140 150 160 170 180
gccctcaggg acctgaagga ggcgcggggg cttctcgcca aagacctgag cgttctggcc
      190 200 210 220 230 240
ctgagggaag gccttggcct cccgcccggc gacgacccca tgctcctcgc ctacctcctg
      250 260 270 280 290 300
gacccttcca acaccacccc cgagggggtg gcccggcgct acggcgggga gtggacggag
      310 320 330 340 350 360
gaggcggggg agcgggccgc cctttccgag aggctcttcg ccaacctgtg ggggaggctt
      370 380 390 400 410 420
gagggggagg agaggctcct ttggctttac cgggaggtgg agaggcccct ttccgctgtc
      430 440 450 460 470 480
ctggcccaca tggaggccac gggggtgcgc ctggacgtgg cctatctcag ggccttgtcc
      490 500 510 520 530 540
ctggaggtgg ccgaggagat cgcccgcctc gaggccgagg tcttccgcct ggccggccac
      550 560 570 580 590 600
cccttcaacc tcaactcccg ggaccagctg gaaagggtcc tctttgacga gctagggctt
      610 620 630 640 650 660
cccgccatcg gcaagacgga gaagaccggc aagcgctcca ccagcgccgc cgtcctggag
      670 680 690 700 710 720
gccctccgcg aggcccaccc catcgtggag aagatcctgc agtaccggga gctcaccaag
      730 740 750 760 770 780
ctgaagagca cctacattga ccccttgccg gacctcatcc accccaggac gggccgcctc
      790 800 810 820 830 840
cacacccgct tcaaccagac ggccacggcc acgggcaggc taagtagctc cgatcccaac
      850 860 870 880 890 900
ctccagaaca tccccgtccg caccccgctt gggcagagga tccgccgggc cttcatcgcc
      910 920 930 940 950 960
gaggaggggt ggctattggt ggccctggac tatagccaga tagagctcag ggtgctggcc
      970 980 990 1000 1010 1020
cacctctccg gcgacgagaa cctgatccgg gtcttccagg aggggcggga catccacacg
      1030 1040 1050 1060 1070 1080
gagaccgcca gctggatgtt cggcgtcccc cgggaggccg tggaccccct gatgcgccgg
      1090 1100 1110 1120 1130 1140
gcggccaaga ccatcaactt cggggtcctc tacggcatgt cggcccaccg cctctcccag
      1150 1160 1170 1180 1190 1200
gagctagcca tcccttacga ggaggcccag gccttcattg agcgctactt tcagagcttc
      1210 1220 1230 1240 1250 1260
cccaaggtgc gggcctggat tgagaagacc ctggaggagg gcaggaggcg ggggtacgtg
      1270 1280 1290 1300 1310 1320
gagaccctct tcggccgccg ccgctacgtg ccagacctag aggcccgggt gaagagcgtg
      1330 1340 1350 1360 1370 1380
cgggaggcgg ccgagcgcat ggccttcaac atgcccgtcc agggcaccgc cgccgacctc
      1390 1400 1410 1420 1430 1440
atgaagctgg ctatggtgaa gctcttcccc aggctggagg aaatgggggc caggatgctc
      1450 1460 1470 1480 1490 1500
cttcaggtcc acgacgagct ggtcctcgag gccccaaaag agagggcgga ggccgtggcc
      1510 1520 1530 1540 1550 1560
cggctggcca aggaggtcat ggagggggtg tatcccctgg ccgtgcccct ggaggtggag
      1570 1580 1590 1600 1610 1620
gtggggatag gggaggactg gctctccgcc aaggagtagt aagcttatcg atgata
      1630 1640 1650 1660 1670 1680
```

¹The underlined triplet codons of Klentaq-278 (SEQ ID NO:1) encode amino acids E-358, I-439, and E-440 of Klentaq-278 (SEQ ID NO:2).

TABLE 2
Amino acid sequence of Klentaq-278 (SEQ ID NO:2)¹

```
MGLLHEFGLL ESPKALEEAP WPPPEGAFVG FVLSRKEPMW ADLLALAAAR GGRVHRAPEP
1 10 20 30 40 50 60
YKALRDLKEA RGLLAKDLSV LALREGLGLP PGDDPMLLAY LLDPSNTTPE GVARRYGGEW
                70 80 90 100 120 130
TEEAGERAAL SERLFANLWG RLEGEERLLW LYREVERPLS AVLAHMEATG VRLDVAYLRA
              140 150 160 170 180 190
LSLEVAEEIA RLEAEVFRLA GHPFNLNSRD QLERVLFDEL GLPAIGKTEK TGKRSTSAAV
              200 210 220 230 240 250
LEALREAHPI VEKILQYREL TKLKSTYIDP LPDLIHPRTG RLHTRFNQTA TATGRLSSSD
              260 270 280 290 300 310
PNLQNIPVRT PLGQRIRRAF IAEEGWLLVA LDYSQIELRV LAHLSGDENL IRVFQEGRDI
              320 330 340 350 360 370
HTETASWMFG VPREAVDPLM RRAAKTINFG VLYGMSAHRL SQELAIPYEE AQAFIERYFQ
              380 390 400 410 420 430
SFPKVRAWIE KTLEEGRRRG YVETLFGRRR YVPDLEARVK SVREAAERMA FNMPVQGTAA
              440 450 460 470 480 490
DLMKLAMVKL FPRLEEMGAR MLLQVHDELV LEAPKERAEA VARLAKEVME GVYPLAVPLE
              500 510 520 530 540 550
VEVGIGEDWL SAKE
              560
```

¹The underlined amino acid residues, E-358, I-439, and E-440, of Klentaq-278 (SEQ ID NO:2), correspond to E-626, I-707, and E-708, respectively, of the full-length wild-type Taq DNA polymerase.

TABLE 3
Nucleic acid sequence of KT-6 (SEQ ID NO:3)¹

```
atgggg
1
ctcctccacg agttcggcct tctggaaagc cccaaggccc tggaggaggc cccctggccc
      10 20 30 40 50 60
ccgccggaag gggccttcgt gggctttgtg ctttcccgca aggagcccat gtgggccgat
      70 80 90 100 110 120
cttctggccc tggccgccgc cagggggggc cgggtccacc gggcccccga gccttataaa
      130 140 150 160 170 180
gccctcaggg acctgaagga ggcgcggggg cttctcgcca aagacctgag cgttctggcc
      190 200 210 220 230 240
ctgagggaag gccttggcct cccgcccggc gacgacccca tgctcctcgc ctacctcctg
      250 260 270 280 290 300
gacccttcca acaccacccc cgagggggtg gcccggcgct acggcgggga gtggacggag
      310 320 330 340 350 360
gaggcggggg agcgggccgc cctttccgag aggctcttcg ccaacctgtg ggggaggctt
      370 380 390 400 410 420
gagggggagg agaggctcct ttggctttac cgggaggtgg agaggcccct ttccgctgtc
      430 440 450 460 470 480
ctggcccaca tggaggccac gggggtgcgc ctggacgtgg cctatctcag ggccttgtcc
      490 500 510 520 530 540
ctggaggtgg ccgaggagat cgcccgcctc gaggccgagg tcttccgcct ggccggccac
      550 560 570 580 590 600
cccttcaacc tcaactcccg ggaccagctg gaaagggtcc tctttgacga gctagggctt
      610 620 630 640 650 660
cccgccatcg gcaagacgga gaagaccggc aagcgctcca ccagcgccgc cgtcctggag
      670 680 690 700 710 720
gccctccgcg aggcccaccc catcgtggag aagatcctgc agtaccggga gctcaccaag
      730 740 750 760 770 780
ctgaagagca cctacattga ccccttgccg gacctcatcc accccaggac gggccgcctc
      790 800 810 820 830 840
cacacccgct tcaaccagac ggccacggcc acgggcaggc taagtagctc cgatcccaac
      850 860 870 880 890 900
ctccagaaca tccccgtccg caccccgctt gggcagagga tccgccgggc cttcatcgcc
      910 920 930 940 950 960
gaggaggggt ggctattggt ggccctggac tatagccaga tagagctcag ggtgctggcc
      970 980 990 1000 1010 1020
cacctctccg gcgacgagaa cctgatccgg gtcttccagg aggggcggga catccacacg
      1030 1040 1050 1060 1070 1080
gagaccgcca gctggatgtt cggcgtcccc cgggaggccg tggaccccct gatgcgccgg
      1090 1100 1110 1120 1130 1140
gcggccaaga ccatcaactt cggggtcctc tacggcatgt cggcccaccg cctctcccag
      1150 1160 1170 1180 1190 1200
gagctagcca tcccttacga ggaggcccag gccttcattg agcgctactt tcagagcttc
      1210 1220 1230 1240 1250 1260
cccaaggtgc gggcctggct tgtgaagacc ctggaggagg gcaggaggcg ggggtacgtg
      1270 1280 1290 1300 1310 1320
gagaccctct tcggccgccg ccgctacgtg ccagacctag aggcccgggt gaagagcgtg
      1330 1340 1350 1360 1370 1380
cgggaggcgg ccgagcgcat ggccttcaac atgcccgtcc agggcaccgc cgccgacctc
      1390 1400 1410 1420 1430 1440
atgaagctgg ctatggtgaa gctcttcccc aggctggagg aaatgggggc caggatgctc
      1450 1460 1470 1480 1490 1500
cttcaggtcc acgacgagct ggtcctcgag gccccaaaag agagggcgga ggccgtggcc
      1510 1520 1530 1540 1550 1560
cggctggcca aggaggtcat ggagggggtg tatcccctgg ccgtgcccct ggaggtggag
      1570 1580 1590 1600 1610 1620
gtggggatag gggaggactg gctctccgcc aaggagtagt aagcttatcg atgata
      1630 1640 1650 1660 1670 1680
```

¹The underlined triplet codons of KT-6 (SEQ ID NO:3) encode amino acids E-358, L-439, and V-440 of KT-6 (SEQ ID NO:4).

TABLE 4
Amino acid sequence of KT-6 (SEQ ID NO:4)¹

```
MGLLHEFGLL ESPKALEEAP WPPPEGAFVG FVLSRKEPMW ADLLALAAAR GGRVHRAPEP
1 10 20 30 40 50 60
YKALRDLKEA RGLLAKDLSV LALREGLGLP PGDDPMLLAY LLDPSNTTPE GVARRYGGEW
                70 80 90 100 120 130
TEEAGERAAL SERLFANLWG RLEGEERLLW LYREVERPLS AVLAHMEATG VRLDVAYLRA
              140 150 160 170 180 190
LSLEVAEEIA RLEAEVFRLA GHPFNLNSRD QLERVLFDEL GLPAIGKTEK TGKRSTSAAV
              200 210 220 230 240 250
LEALREAHPI VEKILQYREL TKLKSTYIDP LPDLIHPRTG RLHTRFNQTA TATGRLSSSD
              260 270 280 290 300 310
PNLQNIPVRT PLGQRIRRAF IAEEGWLLVA LDYSQIELRV LAHLSGDENL IRVFQEGRDI
              320 330 340 350 360 370
HTETASWMFG VPREAVDPLM RRAAKTINFG VLYGMSAHRL SQELAIPYEE AQAFIERYFQ
              380 390 400 410 420 430
SFPKVRAWLV KTLEEGRRRG YVETLFGRRR YVPDLEARVK SVREAAERMA FNMPVQGTAA
              440 450 460 470 480 490
DLMKLAMVKL FPRLEEMGAR MLLQVHDELV LEAPKERAEA VARLAKEVME GVYPLAVPLE
              500 510 520 530 540 550
VEVGIGEDWL SAKE
              560
```

¹The underlined amino acid residues of KT-6 (SEQ ID NO:4) correspond to amino acids E-358, L-439, and V-440.

TABLE 5
Nucleic acid sequence of KT-7 (SEQ ID NO:5)¹

```
atgggg
1
ctcctccacg agttcggcct tctggaaagc cccaaggccc tggaggaggc cccctggccc
      10 20 30 40 50 60
ccgccggaag gggccttcgt gggctttgtg ctttcccgca aggagcccat gtgggccgat
      70 80 90 100 110 120
cttctggccc tggccgccgc cagggggggc cgggtccacc gggcccccga gccttataaa
      130 140 150 160 170 180
gccctcaggg acctgaagga ggcgcggggg cttctcgcca aagacctgag cgttctggcc
      190 200 210 220 230 240
ctgagggaag gccttggcct cccgcccggc gacgacccca tgctcctcgc ctacctcctg
      250 260 270 280 290 300
gacccttcca acaccacccc cgagggggtg gcccggcgct acggcgggga gtggacggag
      310 320 330 340 350 360
gaggcggggg agcgggccgc cctttccgag aggctcttcg ccaacctgtg ggggaggctt
      370 380 390 400 410 420
gagggggagg agaggctcct ttggctttac cgggaggtgg agaggcccct ttccgctgtc
      430 440 450 460 470 480
ctggcccaca tggaggccac gggggtgcgc ctggacgtgg cctatctcag ggccttgtcc
      490 500 510 520 530 540
ctggaggtgg ccgaggagat cgcccgcctc gaggccgagg tcttccgcct ggccggccac
      550 560 570 580 590 600
cccttcaacc tcaactcccg ggaccagctg gaaagggtcc tctttgacga gctagggctt
      610 620 630 640 650 660
cccgccatcg gcaagacgga gaagaccggc aagcgctcca ccagcgccgc cgtcctggag
      670 680 690 700 710 720
gccctccgcg aggcccaccc catcgtggag aagatcctgc agtaccggga gctcaccaag
      730 740 750 760 770 780
ctgaagagca cctacattga ccccttgccg gacctcatcc accccaggac gggccgcctc
      790 800 810 820 830 840
cacacccgct tcaaccagac ggccacggcc acgggcaggc taagtagctc cgatcccaac
      850 860 870 880 890 900
ctccagaaca tccccgtccg caccccgctt gggcagagga tccgccgggc cttcatcgcc
      910 920 930 940 950 960
gaggaggggt ggctattggt ggccctggac tatagccaga tagagctcag ggtgctggcc
      970 980 990 1000 1010 1020
cacctctccg gcgacaagaa cctgatccgg gtcttccagg aggggcggga catccacacg
      1030 1040 1050 1060 1070 1080
gagaccgcca gctggatgtt cggcgtcccc cgggaggccg tggaccccct gatgcgccgg
      1090 1100 1110 1120 1130 1140
gcggccaaga ccatcaactt cggggtcctc tacggcatgt cggcccaccg cctctcccag
      1150 1160 1170 1180 1190 1200
gagctagcca tcccttacga ggaggcccag gccttcattg agcgctactt tcagagcttc
      1210 1220 1230 1240 1250 1260
cccaaggtgc gggcctggct tgtgaagacc ctggaggagg gcaggaggcg ggggtacgtg
      1270 1280 1290 1300 1310 1320
gagaccctct tcggccgccg ccgctacgtg ccagacctag aggcccgggt gaagagcgtg
      1330 1340 1350 1360 1370 1380
cgggaggcgg ccgagcgcat ggccttcaac atgcccgtcc agggcaccgc cgccgacctc
      1390 1400 1410 1420 1430 1440
atgaagctgg ctatggtgaa gctcttcccc aggctggagg aaatgggggc caggatgctc
      1450 1460 1470 1480 1490 1500
cttcaggtcc acgacgagct ggtcctcgag gccccaaaag agagggcgga ggccgtggcc
      1510 1520 1530 1540 1550 1560
cggctggcca aggaggtcat ggagggggtg tatcccctgg ccgtgcccct ggaggtggag
      1570 1580 1590 1600 1610 1620
gtggggatag gggaggactg gctctccgcc aaggagtagt aagcttatcg atgata
      1630 1640 1650 1660 1670 1680
```

¹The underlined triplet codons of KT-7 (SEQ ID NO:5) encode amino acids K-358, L-439, and W-440, of KT-7 (SEQ ID NO:6).

TABLE 6
Amino acid sequence of KT-7 (SEQ ID NO:6)¹

```
MGLLHEFGLL ESPKALEEAP WPPPEGAFVG FVLSRKEPMW ADLLALAAAR GGRVHRAPEP
1 10 20 30 40 50 60
YKALRDLKEA RGLLAKDLSV LALREGLGLP PGDDPMLLAY LLDPSNTTPE GVARRYGGEW
                70 80 90 100 120 130
TEEAGERAAL SERLFANLWG RLEGEERLLW LYREVERPLS AVLAHMEATG VRLDVAYLRA
              140 150 160 170 180 190
LSLEVAEEIA RLEAEVFRLA GHPFNLNSRD QLERVLFDEL GLPAIGKTEK TGKRSTSAAV
              200 210 220 230 240 250
LEALREAHPI VEKILQYREL TKLKSTYIDP LPDLIHPRTG RLHTRFNQTA TATGRLSSSD
              260 270 280 290 300 310
PNLQNIPVRT PLGQRIRRAF IAEEGWLLVA LDYSQIELRV LAHLSGDKNL IRVFQEGRDI
              320 330 340 350 360 370
HTETASWMFG VPREAVDPLM RRAAKTINFG VLYGMSAHRL SQELAIPYEE AQAFIERYFQ
              380 390 400 410 420 430
SFPKVRAWLW KTLEEGRRRG YVETLFGRRR YVPDLEARVK SVREAAERMA FNMPVQGTAA
              440 450 460 470 480 490
DLMKLAMVKL FPRLEEMGAR MLLQVHDELV LEAPKERAEA VARLAKEVME GVYPLAVPLE
              500 510 520 530 540 550
VEVGIGEDWL SAKE
              560
```

¹The underlined amino acid residues of KT-7 (SEQ ID NO:6) correspond to amino acids K-358, L-439, and W-440.

TABLE 7
Nucleic acid sequences of oligonucleotides

| | | |
|---|---|---|
| SEQ ID NO.: | Name | Primer Sequence (5′→3′) |

| | | |
|---|---|---|
| 7 | CCR5 − KOZ | TGGAACAAGATGGATTATCAAGTGTCAAGTCCA |
| | | 1 10 20 30 |
| 8 | CCR5 − 2kb | AGAAGAGCTGAGACATCCGTTCCCCTACAAGAA |
| | | 1 10 20 30 |
| 9 | ccr5 + 1kb | AGGCTGTGTATGAAAACTAAGCCATGTGCACAA |
| | | 1 10 20 30 |
| 10 | ccr5deltaRT | GCAGCGGCAGGACCAGCCCCAAGATGACTATCT |
| | | 1 10 20 30 |
| 11 | KT1 | GAGCCATGGTCCTCCTCCACGAGTTCGGCCTTCTGG |
| | | 1 10 20 30 |
| 12 | RevTaqH | CGGTCCGAAAGCTTCTATCACTCCTTGGCGG |
| | | 1 10 20 30 |
| 13 | DMDex21f | GGCTGTGATAGAGGCTTGTCTATA |
| | | 1 10 20 |
| 14 | DMDex21r | CTGGCCTGCACATCAGAAAAGACT |
| | | 1 10 20 |
| 15 | CCR5 − D5 | AGGTACCTGGCTGTCGTCCATGCTGTGTTT |
| | | 1 10 20 30 |
| 16 | CCR5 − D3 | GATGATGGGGTTGATGCAGCAGTGCGTCAT |
| | | 1 10 20 30 |

### REFERENCES

1. Lantz P-G, Al-Soud W A, Knutsson R, Hahn-Hägerdal B, Radstrom P. 2000. Biotechnical use of the polymerase chain reaction for microbial analysis of biological samples, p. 87-130. In M. R. El-Gewely (ed.), BIOTECHNOLOGY ANNUAL REVIEW, vol. 5. (Elsevier Science B.V., Amsterdam, The Netherlands).
2. Altwegg M, Verhoef J. 1995. Amplification methods in diagnostic microbiology. J Microbiol. Methods 23:3-138.
3. Al-Soud W A, Radstrom P. 2000. Effect of amplification facilitators on diagnostic PCR in the presence of blood, feces and meat. J. Clin. Microbiol. 38: 4463-70.
4. Al-Soud A W, Jönsson L J, Radstrom P. 2000. Identification and characterization of immunoglobulin G in blood as a major inhibitor of diagnostic PCR. J. Clin. Microbiol. 38:345-50.
5. de Franchis R, Cross N C P, Foulkes N S, Cox T M. 1988. A potent inhibitor of Taq polymerase copurifies with human genomic DNA. Nucleic Acids Res. 16:10355.
6. Al-Soud A W, Radstrom P. 1998. Capacity of nine thermostable DNA polymerases to mediate DNA amplification in the presence of PCR-inhibiting samples. Appl. Environ. Microbiol. 64:3748-53.
7. Al-Soud W A, Radstrom P. 2001. Purification ands characterization of PCR-inhibitory components in blood cells. J. Clin. Microbiol. 39:485-93.
8. Frackman S, Kobs G, Simpson D, Storts D. 1998. Betaine and DMSO: enhancing agents for PCR. Promega Notes 65:27.
9. Topal M D, Sinha N K. 1983. Products of bacteriophage T4 genes 32 and 45 improve the accuracy of DNA replication in vitro. J. Biol. Chem. 258:12274-79.
10. Akane A, Matsubara K, Nakamura H, Takahashi S, Kimura K. 1994. Identification of the heme compound copurified with deoxyribonucleic acid (DNA) from bloodstains, a major inhibitor of polymerase chain reaction (PCR) amplification. J Forensic Sci. 39:362-72.
11. Kreader C A. 1996. Relief of amplification inhibition in PCR with bovine serum albumin or T4 gene 32 protein. Appl. Environ. Microbiol. 62:1102-06.
12. Morata P, Queipo-Ortuno I, Colmenero J. 1998. Strategy for optimmizing DNA amplification in a peripheral blood PCR assay used for diagnosis of human brucellosis. J. Clin. Microbiol. 36: 2443-46.
13. Rossen L, Nøskov P, Holmstrøm K, Rasmussen O F. 1992. Inhibition of PCR by components of food samples, microbial diagnostic assays and DNA-extraction solution. Int. J. Food Microbiol. 17:37-45.
14. Izraeli S, Pfleiderer C, Lion T. 1991. Detection of gene expression by PCR amplification of RNA derived from frozen heparinized whole blood. Nucleic Acids Res. 19:6051.
15. Wilson I G 1997. Inhibition and facilitation of nucleic acid amplification. Appl. Environ. Microbiol. 63:3741-51.
16. Al-Soud A W, Lantz P-G, Bäckman A, Olcen P, Radstrom P. 1998. A sample preparation method which facilitates detection of bacteria in blood cultures by the polymerase chain reaction. J. Microbiol. Methods 32:217-224.
17. Klein A, Barsuk R, Dagan S, Nusbaum O, Shouval D, Galun E. 1997. Comparison of methods for extraction of nucleic acid from hemolytic serum for PCR amplification of hepatitis B virus DNA sequences. J. Clin. Microbiol. 35:1897-99.
18. Cattaneo C, Graig O E, James N T, Bolton H. 1997. Comparison of three DNA extraction methods on bone and blood stains up to 43 years old and amplification of three different gene sequences. J. Forensic Sci. 42:1126-35.
19. Bourke M T, Scherczinger C A, Ladd C, Lee H C 1999. NaOH treatment to neutralize inhibitors of Taq polymerase. J. Forensic Sci. 44:1046-50.
20. Kox L F, Rhienthong D, Miranda A M, Udomsantisuk N, Ellis K, van Leeuwven J, van Heusden S, Kuijper S, Kolk A H 1994. A more reliable PCR for detection of Mycobacterium tuberculosis in clinical samples. J. Clin. Microbiol. 32:672-80.
21. Kramvis A, Bukovzer S, Kew M C 1996. Comparison of hepatitis B virus DNA extractions from serum by the QIAamp blood kit, Genereleaser, and the phenol-chloroform method. J. Clin. Microbiol. 34:2731-33.
22. Barnes W M 1992. The fidelity of taq polymerase catalyzing PCR is improved by an N-terminal deletion. Gene 112:29-35.
23. Kermekchiev M B, Tzekov A, Barnes W M 2003. Cold-sensitive mutants of Taq DNA polymerase provide a hot start PCR. Nucleic Acids Res. 31:6139-47.
24. Tabor S, Richardson C C 1995. A single residue in DNA polymerases of the E. coli DNA polymerase I family is critical for distinguishing between deoxy- and dideoxyribonucleotides. Proc. Natl. Acad. Sci., USA 92:6339-43.
25. Tawfik D S, Griffiths A D 1998. Man-made cell-like compartments for molecular evolution. Nature Biotech. 16:652-56.
26. Ghadessy F J, Ong J L, Holliger P. 2001. Direct evolution of polymerase function by compartmentalized self-replication. Proc. Natl. Acad. Sci., USA 98:4552-57.
27. Barnes W M 1994. PCR amplification of up to 35 kb DNA with high fidelity and high yield from bacteriophage templates. Proc. Natl. Acad. Sci., USA 91:2216-20.
28. Barnes W M 1994. Tips and tricks for long and accurate PCR. TIBS 19:342-46.
29. Ausubel F M, Brent R, Kingston R E, Moore D D et al. 1987. CURRENT PROTOCOLS IN MOLECULAR BIOLOGY. John Wiley & Sons, New York.
30. Sambrook J 1989. Molecular cloning: a laboratory manual. Cold Spring Harbor Laboratory, Cold Spring Harbor.
31. Barnes W M. Jul. 25, 1995. U.S. Pat. No. 5,436,149, Thermostable DNA polymerase with enhanced thermostability and enhanced length and efficiency of primer extension.
32. Barnes W M. Apr. 1, 1997. U.S. Pat. No. 5,616,494, Thermus aquaticus DNA polymerase lacking the n-terminal 235 amino acids of taq DNA polymerase.
33. Scalice E R, Sharkey D J, Daiss J L. 1994. Monoclonal antibodies prepared against the DNA polymerase from Thermus aquaticus are potent inhibitors of enzyme activity. J. Immunol. Methods 172:147-63.
34. Sharkey D J, Scalice E R, Christy K G Jr, Atwood S M, Daiss J L. 1994. Antibodies as thermolabile switches: high temperature triggering for the polymerase chain reaction. Biotechnology 12:506-9.
35. Kellogg D E, Rybalkin I, Chen S, Mukhamedova N, Vlasik T, Siebert P D, Chenchik A. 1994. TaqStart Antibody: “hot start” PCR facilitated by a neutralizing monoclonal antibody directed against Taq DNA polymerase. Biotechniques 16:1134-7.
36. Baskaran N, Kandpal R P, Bhargava A K, Glynn M W, Bale A, Weissman S M. 1996. Uniform amplification of a mixture of deoxyribonucleic acids with varying GC content. Genome Res. 6:633-8.

1. A method of obtaining DNA amplification of a nucleic acid target from a volume of whole blood comprising performing DNA amplification in a PCR assay mixture with a blood-resistant polymerase.

2. The method of claim 1, wherein the blood-resistant polymerase comprises at least one member selected from the grouping consisting of Klentaq1 (SEQ ID NO:2), KT-6 (SEQ ID NO:4), and KT-7 (SEQ ID NO:6).

3. The method of claim 1, wherein the volume of whole blood comprises greater than about 1% of the PCR assay mixture.

4. The method of claim 1, wherein the volume of whole blood comprises greater than about 3% of the PCR assay mixture.

5. The method of claim 1, wherein the volume of whole blood comprises a range from about 5% to about 20% of the PCR assay mixture.

6. The method of claim 1, wherein the volume of whole blood comprises about 10% of the PCR assay mixture.

7. A method of obtaining DNA amplification of a nucleic acid target from whole blood, comprising:
adding a first solution comprising a DNA amplification cocktail into a reaction vessel;
underlayering the first solution with a volume of whole blood to yield a final volume of reaction; and
performing a thermal cycling program to effect DNA amplification,
wherein the DNA amplification cocktail comprises a blood-resistant polymerase.

8. The method of claim 7, wherein the volume of whole blood comprises greater than about 1% of the final volume of reaction.

9. The method of claim 7, wherein the volume of whole blood comprises greater than about 3% of the final volume of reaction.

10. The method of claim 7, wherein the volume of whole blood comprises a range from about 5% to about 20% of the final volume of reaction.

11. The method of claim 7, wherein the volume of whole blood comprises about 10% of the final volume of reaction.

12. The method of claim 7, wherein the blood-resistant polymerase is a polypeptide selected from the group consisting of Klentaq1 (SEQ ID NO:2), KT-6 (SEQ ID NO:4), and KT-7 (SEQ ID NO:6).

13. The method of claim 7 wherein the blood-resistant polymerase comprises KT-1 (SEQ ID NO:2).

14. The method of claim 7 wherein the blood-resistant polymerase comprises KT-6 (SEQ ID NO:4).

15. The method of claim 7 wherein the blood-resistant polymerase comprises KT-7 (SEQ ID NO:6).

16. A method of obtaining a hot start for DNA amplification of a nucleic acid target comprising:
preparation of the reaction cocktail comprising at least a first volume component and a second volume component,
wherein the second volume component is heavier than the first volume component,
wherein the first volume component comprises a DNA polymerase cocktail lacking an essential constituent required for DNA amplification activity,
wherein the second volume component comprises the essential constituent required for DNA amplification activity, and
wherein the second volume component is underlayed below the first volume component without undue mixing before a DNA amplification reaction is initiated.

17. The method of claim 16, wherein the second volume component further comprises a solution comprising a magnesium salt and deoxynucleotide triphosphates.

18. The method of claim 16, wherein the second volume component further comprises a target template nucleic acid.

19. The method of claim 16 wherein the second volume component further comprises a first DNA polymerase enzyme.

20. The method of claim 16 wherein the second volume component further comprises sucrose or sorbitol.

21. The method of claim 16 wherein the second volume component further comprises betaine.

22. The method of claim 16 wherein the second volume component further comprises a dense composition.

23. The method of claim 16 wherein the second volume component further comprises whole blood or a subfraction thereof.

24. An isolated polypeptide comprising an amino acid sequence comprising at least 80% amino acid sequence identity with SEQ ID NO:2, wherein the isolated polypeptide comprises a blood-resistant polymerase.

25. The isolated polypeptide of claim 24, wherein the amino acid sequence comprises at least 85% amino acid sequence identity with SEQ ID NO:2.

26. The isolated polypeptide of claim 24, wherein the amino acid sequence comprises at least 90% amino acid sequence identity with SEQ ID NO:2.

27. The isolated polypeptide of claim 24, wherein the amino acid sequence comprises at least 95% amino acid sequence identity with SEQ ID NO:2.

28. The isolated polypeptide of claim 24, wherein the amino acid sequence comprises at least 98% amino acid sequence identity with SEQ ID NO:2.

29. The isolated polypeptide of claim 24, wherein the amino acid sequence comprises at least 99% amino acid sequence identity with SEQ ID NO:2.

30. The isolated polypeptide of claim 24, wherein the amino acid sequence comprises SEQ ID NO:2 having at least 2 amino acid substitutions.

31. An isolated polypeptide comprising an amino acid sequence comprising at least 80% amino acid sequence identity with SEQ ID NO:4, wherein the isolated polypeptide comprises a blood-resistant polymerase.

32. The isolated polypeptide of claim 31, wherein the amino acid sequence comprises at least 85% amino acid sequence identity with SEQ ID NO:4.

33. The isolated polypeptide of claim 31, wherein the amino acid sequence comprises at least 90% amino acid sequence identity with SEQ ID NO:4.

34. The isolated polypeptide of claim 31, wherein the amino acid sequence comprises at least 95% amino acid sequence identity with SEQ ID NO:4.

35. The isolated polypeptide of claim 31, wherein the amino acid sequence comprises at least 98% amino acid sequence identity with SEQ ID NO:4.

36. The isolated polypeptide of claim 31, wherein the amino acid sequence comprises at least 99% amino acid sequence identity with SEQ ID NO:4.

37. The isolated polypeptide of claim 31, wherein the amino acid sequence comprises SEQ ID NO:4.

38. An isolated polypeptide comprising an amino acid sequence comprising at least 80% amino acid sequence identity with SEQ ID NO:6, wherein the isolated polypeptide comprises a blood-resistant polymerase.

39. The isolated polypeptide of claim 38, wherein the amino acid sequence comprises at least 85% amino acid sequence identity with SEQ ID NO:6.

40. The isolated polypeptide of claim 38, wherein the amino acid sequence comprises at least 90% amino acid sequence identity with SEQ ID NO:6.

41. The isolated polypeptide of claim 38, wherein the amino acid sequence comprises at least 95% amino acid sequence identity with SEQ ID NO:6.

42. The isolated polypeptide of claim 38, wherein the amino acid sequence comprises at least 98% amino acid sequence identity with SEQ ID NO:6.

43. The isolated polypeptide of claim 38, wherein the amino acid sequence comprises at least 99% amino acid sequence identity with SEQ ID NO:6.

44. The isolated polypeptide of claim 38, wherein the amino acid sequence comprises SEQ ID NO:6.

45. An isolated nucleic acid comprising a nucleotide sequence comprising at least 80% nucleotide sequence identity of SEQ ID NO:1, wherein the isolated nucleic acid encodes a blood-resistant polymerase.

46. The isolated nucleic acid of claim 45, wherein the nucleotide sequence comprises at least 85% nucleotide sequence identity of SEQ ID NO:1.

47. The isolated nucleic acid of claim 45, wherein the nucleotide sequence comprises at least 90% nucleotide sequence identity of SEQ ID NO:1.

48. The isolated nucleic acid of claim 45, wherein the nucleotide sequence comprises at least 95% nucleotide sequence identity of SEQ ID NO:1.

49. The isolated nucleic acid of claim 45, wherein the nucleotide sequence comprises at least 98% nucleotide sequence identity of SEQ ID NO:1.

50. The isolated nucleic acid of claim 45, wherein the nucleotide sequence comprises at least 99% nucleotide sequence identity of SEQ ID NO:1.

51. The isolated nucleic acid of claim 45, wherein the nucleotide sequence comprises SEQ ID NO:1 having at least 2 codon substitutions.

52. An isolated nucleic acid comprising a nucleotide sequence comprising at least 80% nucleotide sequence identity of SEQ ID NO:3, wherein the isolated nucleic acid encodes a blood-resistant polymerase.

53. The isolated nucleic acid of claim 52, wherein the nucleotide sequence comprises at least 85% nucleotide sequence identity of SEQ ID NO:3.

54. The isolated nucleic acid of claim 52, wherein the nucleotide sequence comprises at least 90% nucleotide sequence identity of SEQ ID NO:3.

55. The isolated nucleic acid of claim 52, wherein the nucleotide sequence comprises at least 95% nucleotide sequence identity of SEQ ID NO:3.

56. The isolated nucleic acid of claim 52, wherein the nucleotide sequence comprises at least 98% nucleotide sequence identity of SEQ ID NO:3.

57. The isolated nucleic acid of claim 52, wherein the nucleotide sequence comprises at least 99% nucleotide sequence identity of SEQ ID NO:3.

58. The isolated nucleic acid of claim 52, wherein the nucleotide sequence comprises SEQ ID NO:3.

59. An isolated nucleic acid comprising a nucleotide sequence comprising at least 80% nucleotide sequence identity of SEQ ID NO:5, wherein the isolated nucleic acid encodes a blood-resistant polymerase.

60. The isolated nucleic acid of claim 59, wherein the nucleotide sequence comprises at least 85% nucleotide sequence identity of SEQ ID NO:5.

61. The isolated nucleic acid of claim 59, wherein the nucleotide sequence comprises at least 90% nucleotide sequence identity of SEQ ID NO:5.

62. The isolated nucleic acid of claim 59, wherein the nucleotide sequence comprises at least 95% nucleotide sequence identity of SEQ ID NO:5.

63. The isolated nucleic acid of claim 59, wherein the nucleotide sequence comprises at least 98% nucleotide sequence identity of SEQ ID NO:5.

64. The isolated nucleic acid of claim 59, wherein the nucleotide sequence comprises at least 99% nucleotide sequence identity of SEQ ID NO:5.

65. A method of screening for a faster elongating polymerase, comprising:
conducting PCR assays on a nucleic acid target using a thermal cycling program, wherein the thermal cycling program comprises an extension reaction performed for at most about 20 sec per 2 kilobase pair amplicon, and the collection of mutant DNA polymerases to be screened are cold-sensitive.

66. A kit for performing PCR assays on samples of whole blood, comprising a blood-resistant polymerase.

67. The method according to claim 2, further comprising a second DNA polymerase.

68. The method according to claim 67, wherein the second DNA polymerase comprises a DNA polymerase having a 3′-exonuclease activity.

69. The method of claim 68, wherein the second DNA polymerase comprises at least one member selected from the group consisting of Vent DNA polymerase, Deep Vent DNA polymerase, Pfu DNA polymerase, and Pwu DNA polymerase.

70. The method according to claim 19, further comprising a second DNA polymerase.

71. The method according to claim 70, wherein the second DNA polymerase comprises a DNA polymerase having a 3′-exonuclease activity.

72. The method of claim 71, wherein the second DNA polymerase comprises at least one member selected from the group consisting of Vent DNA polymerase, Deep Vent DNA polymerase, Pfu DNA polymerase, and Pwu DNA polymerase.


SEQUENCE LISTING (16 sequences)

SEQ ID NO: 1 - DNA, 1682 bp, Thermus aquaticus
atggggctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacggagaagaccggcaagcgctccaccagcgccgccgtcctggaggccctccgcgaggcccaccccatcgtggagaagatcctgcagtaccgggagctcaccaagctgaagagcacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggagaccgccagctggatgttcggcgtcccccgggaggccgtggaccccctgatgcgccgggcggccaagaccatcaacttcggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggagaccctcttcggccgccgccgctacgtgccagacctagaggcccgggtgaagagcgtgcgggaggcggccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggagtagtaagcttatcgatgata

SEQ ID NO: 2 - protein, 554 aa, Thermus aquaticus
MGLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 3 - DNA, 1682 bp, Thermus aquaticus
atggggctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacggagaagaccggcaagcgctccaccagcgccgccgtcctggaggccctccgcgaggcccaccccatcgtggagaagatcctgcagtaccgggagctcaccaagctgaagagcacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggagaccgccagctggatgttcggcgtcccccgggaggccgtggaccccctgatgcgccgggcggccaagaccatcaacttcggggtcctctacggcatgtcggcccaccgcctctcccaggagctagccatcccttacgaggaggcccaggccttcattgagcgctactttcagagcttccccaaggtgcgggcctggcttgtgaagaccctggaggagggcaggaggcgggggtacgtggagaccctcttcggccgccgccgctacgtgccagacctagaggcccgggtgaagagcgtgcgggaggcggccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggagtagtaagcttatcgatgata

SEQ ID NO: 4 - protein, 554 aa, Thermus aquaticus
MGLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWLVKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 5 - DNA, 1682 bp, Thermus aquaticus
atggggctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacggagaagaccggcaagcgctccaccagcgccgccgtcctggaggccctccgcgaggcccaccccatcgtggagaagatcctgcagtaccgggagctcaccaagctgaagagcacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacaagaacctgatccgggtcttccaggaggggcgggacatccacacggagaccgccagctggatgttcggcgtcccccgggaggccgtggaccccctgatgcgccgggcggccaagaccatcaacttcggggtcctctacggcatgtcggcccaccgcctctcccaggagctagccatcccttacgaggaggcccaggccttcattgagcgctactttcagagcttccccaaggtgcgggcctggcttgtgaagaccctggaggagggcaggaggcgggggtacgtggagaccctcttcggccgccgccgctacgtgccagacctagaggcccgggtgaagagcgtgcgggaggcggccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggagtagtaagcttatcgatgata

SEQ ID NO: 6 - protein, 554 aa, Thermus aquaticus
MGLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDKNLIRVFQEGRDIHTETASWMFGVPREAVDPLMRRAAKTINFGVLYGMSAHRLSQELAIPYEEAQAFIERYFQSFPKVRAWLWKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 7 - DNA, 33 bp, Artificial Sequence
  Description of Artificial Sequence Synthetic oligonucleotide
tggaacaagatggattatcaagtgtcaagtcca

SEQ ID NO: 8 - DNA, 33 bp, Artificial Sequence
  Description of Artificial Sequence Synthetic oligonucleotide
agaagagctgagacatccgttcccctacaagaa

SEQ ID NO: 9 - DNA, 33 bp, Artificial Sequence
  Description of Artificial Sequence Synthetic oligonucleotide
aggctgtgtatgaaaactaagccatgtgcacaa

SEQ ID NO: 10 - DNA, 33 bp, Artificial Sequence
  Description of Artificial Sequence Synthetic oligonucleotide
gcagcggcaggaccagccccaagatgactatct

SEQ ID NO: 11 - DNA, 36 bp, Artificial Sequence
  Description of Artificial Sequence Synthetic oligonucleotide
gagccatggtcctcctccacgagttcggccttctgg

SEQ ID NO: 12 - DNA, 31 bp, Artificial Sequence
  Description of Artificial Sequence Synthetic oligonucleotide
cggtccgaaagcttctatcactccttggcgg

SEQ ID NO: 13 - DNA, 24 bp, Artificial Sequence
  Description of Artificial Sequence Synthetic oligonucleotide
ggctgtgatagaggcttgtctata

SEQ ID NO: 14 - DNA, 24 bp, Artificial Sequence
  Description of Artificial Sequence Synthetic oligonucleotide
ctggcctgcacatcagaaaagact

SEQ ID NO: 15 - DNA, 30 bp, Artificial Sequence
  Description of Artificial Sequence Synthetic oligonucleotide
aggtacctggctgtcgtccatgctgtgttt

SEQ ID NO: 16 - DNA, 30 bp, Artificial Sequence
  Description of Artificial Sequence Synthetic oligonucleotide
gatgatggggttgatgcagcagtgcgtcat